(19) United States
(12) Patent Application Publication
(10) Pub. No.: US 2024/0127935 A1
(43) Pub. Date: Apr. 18, 2024

(54) MEDICAL COMMUNICATION SYSTEM BASED ON INTERNET OF THINGS

(71) Applicant: UNION HOSPITAL TONGJI MEDICAL COLLEGE HUST, Wuhan (CN)

(72) Inventors: Juan TAO, Wuhan (CN); Yan LI, Wuhan (CN); Jinjin ZHU, Wuhan (CN); Liu YANG, Wuhan (CN); Jing YANG, Wuhan (CN)

(73) Assignee: UNION HOSPITAL TONGJI MEDICAL COLLEGE HUST, Wuhan, HB (CN)

(21) Appl. No.: 18/226,734

(22) Filed: Jul. 26, 2023

(30) Foreign Application Priority Data
Oct. 18, 2022 (CN) 2022-11274101.9

(51) Int. Cl. G16H 40/20; G16H 15/00; G16H 40/67

(52) U.S. Cl. CPC G16H 40/20; G16H 15/00; G16H 40/67; G16H 10/60

(57) ABSTRACT

A medical communication system based on an Internet of Things is provided. Medical information monitoring modules are configured to acquire medical relevant information of various departments; an inquiry information monitoring module is configured to acquire patient inquiry information of various patients making an inquiry; a medical communication information module is configured to perform information integration process on the medical relevant information and the patient inquiry information based on the Internet of Things to obtain processed information; a smart ward platform is configured to fuse the processed information to obtain fused information, and perform data interaction with the medical information monitoring modules according to the fused information; and a visual information terminal is configured to extract three-dimensional data in the fused information and perform character modeling to obtain a virtual character model, and perform human-computer interaction according to the virtual character model and the fused information.

### CROSS-REFERENCE TO RELATED APPLICATION

This patent disclosure claims the benefit and priority of Chinese Patent Application No. 202211274101.9, entitled “Medical Communication System Based on Internet of Things”, filed with the China National Intellectual Property Administration on Oct. 18, 2022, the disclosure of which is incorporated by reference herein in its entirety as part of the present disclosure.

### TECHNICAL FIELD

The present disclosure relates to the technical field of medical communications, particularly relates to a medical communication system based on an Internet of Things.

### BACKGROUND

A medical communication system is a core device of a medical system. A good medical communication system can improve the work efficiency of every medical staff and the collaborative work efficiency of all medical staffs, achieving faster diagnosis, treatment, and service for patients.

At present, most hospitals still use fixed-line phones and mobile phones for communication, which makes communications unprofessional and relatively fixed, and lacks flexibility. Although the mobile phones are more flexible in operation compared with the fixed-line phones, they are not perfect enough in voice call and group call functions. When using a mobile phone, a user needs to hold the mobile phone with one hand, which is not conducive to working while being on a call due to the occupied hand, and the collaborative work efficiency is low.

In the prior art, an Internet of Things technology is applied to build a smart medical communication information system, which achieves multi-network integration according to smart ward Internet of Things platform. However, the smart medical communication information system only integrates information of various subsystems and cannot achieve face-to-face communication and intuitive information displaying according to actual situations of a staff, causing inconvenient communication.

### SUMMARY

In order to overcome the shortcomings in the prior art, the present disclosure aims to provide a medical communication system based on an Internet of Things.

In order to achieve the above-mentioned purpose, the present disclosure provides the following solution.

A medical communication system based on an Internet of Things includes:
a plurality of medical information monitoring modules, configured to acquire medical relevant information of various departments;
an inquiry information monitoring module, configured to acquire patient inquiry information of various patients making an inquiry;
a medical communication information module, connected to the medical information monitoring module and the inquiry information monitoring module and configured to perform information integration process on the medical relevant information and the patient inquiry information based on the Internet of Things to obtain processed information;
a smart ward platform, connected to the medical communication information module and configured to fuse the processed information to obtain fused information, and perform data interaction with the medical information monitoring module according to the fused information; and
a visual information terminal, connected to the smart ward platform and configured to extract three-dimensional data in the fused information and perform character modeling to obtain a virtual character model, and perform human-computer interaction according to the virtual character model and the fused information.

In some embodiments, the medical information monitoring module includes a transfusion monitoring unit, an infant anti-theft unit, a physical sign acquisition unit, a staff locating unit, a moving nursing unit, a nursing management unit, a nursing path unit, a ward round unit, a hospital information unit, a laboratory information management unit, a picture archiving and communication unit, and electronic medical records.

In some embodiments, the medical communication information module includes a nurse station, wards, a ward entrance machine, a corridor display screen, a video monitor, and a switch.

In some embodiments, the visual information terminal includes:
an information extraction unit, configured to perform information screening on the fused information to obtain character picture information in the fused information;
a parsing unit, connected to the information extraction unit and configured to perform three-dimensional parsing on the character picture information to obtain three-dimensional data;
a modeling unit, connected to the parsing unit and configured to build the virtual character model according to the three-dimensional data; and
an information interaction unit, connected to the modeling unit and the information extraction unit and configured to perform human-computer interaction according to the virtual character model and the fused information.

According to the specific embodiments provided by the present disclosure, the present disclosure discloses the following technical effects.

The present disclosure provides a medical communication system based on an Internet of Things, including: a plurality of medical information monitoring modules, configured to acquire medical relevant information of various departments; an inquiry information monitoring module, configured to acquire patient inquiry information of various patients making an inquiry; a medical communication information module, connected to the medical information monitoring module and the inquiry information monitoring module and configured to perform information integration process on the medical relevant information and the patient inquiry information based on the Internet of Things to obtain processed information; a smart ward platform, connected to the medical communication information module and configured to fuse the processed information to obtain fused information, and perform data interaction with the medical information monitoring module according to the fused information; and a visual information terminal, connected to the smart ward platform and configured to extract three-dimensional data in the fused information and perform character modeling to obtain a virtual character model, and perform human-computer interaction according to the virtual character model and the fused information. The present disclosure can perform visual displaying and human-computer interaction according to the obtained information, thereby improving the convenience and efficiency of medical communication.

### BRIEF DESCRIPTION OF THE DRAWINGS

In order to describe the embodiments of the present disclosure or technical solutions in the existing art more clearly, drawings required to be used, in the embodiments will be briefly introduced below. Apparently, the drawings in the descriptions below are only some embodiments of the present disclosure. Those ordinarily skilled in the art also can acquire other drawings according to these drawings without creative work.

FIG. 1 is a structural diagram of a system provided by an embodiment of the present disclosure.

### DETAILED DESCRIPTION OF THE EMBODIMENTS

The technical solutions in the embodiments of the present disclosure will be described clearly and completely below in combination with the accompanying drawings of the embodiments of the present disclosure. Apparently, the described embodiments are only part of the embodiments of the present disclosure, not all embodiments. All other embodiments obtained by those of ordinary skills in the art based on the embodiments in the present disclosure without creative work shall fall within the protection scope of the present disclosure.

The “embodiment” mentioned herein means that specific features, structures or characteristics described in combination with the embodiments may be included in at least one embodiment of the present disclosure. This phrase appearing at each position in the description does not refer to the same embodiment, and is not an independent or candidate embodiment mutually exclusive with other embodiments. Those skilled in the art can explicitly and implicitly understand that the embodiments described herein may be combined with other embodiments.

The terms “first”, “second”, “third”, and “fourth” in the specification and claims of the present disclosure and the drawings are used to distinguish different objects, and are not intended to describe a specific order. In addition, the terms “include/comprise”, “have” and any of their variations intend to cover non-exclusive inclusion. For example, an expression of including a series of steps, processes, methods, or the like are not limited to listed steps, but optionally, steps which are not listed are also included, or optionally, other steps which are inherent to these processes, methods, products, or devices are also included.

The present disclosure aims to provide a medical communication system based on an Internet of Things, which can perform visual displaying and human-computer interaction according to the obtained information, thereby improving the convenience and efficiency of medical communication.

In order to make the above-mentioned purposes, characteristics, and advantages of the present disclosure more obvious and understandable, the present disclosure is further described in detail below with reference to the accompanying drawings and specific implementations.

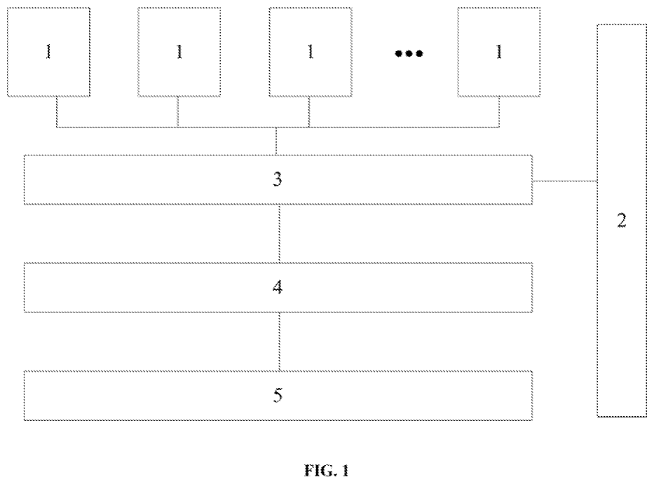

FIG. 1 is a structural diagram of a system provided by an embodiment of the present disclosure. As shown in FIG. 1, the present disclosure provides a medical communication system based on an Internet of Things, including:
a plurality of medical information monitoring modules 1, configured to acquire medical relevant information of various departments;
an inquiry information monitoring module 2, configured to acquire patient inquiry information of various patients making an inquiry;
a medical communication information module 3, connected to the medical information monitoring modules 1 and the inquiry information monitoring module and configured to perform information integration process on the medical relevant information and the patient inquiry information based on the Internet of Things to obtain processed information;
a smart ward platform 4, connected to the medical communication information module 3 and configured to fuse the processed information to obtain fused information, and perform data interaction with the medical information monitoring modules 1 according to the fused information; and
a visual information terminal 5, connected to the smart ward platform 4 and configured to extract three-dimensional data in the fused information and perform character modeling to obtain a virtual character model, and perform human-computer interaction according to the virtual character model and the fused information.

References in the Drawings refer to aspects of the present invention as follows:
1: a medical information monitoring module;
2: inquiry information monitoring module;
3: medical communication information module;
4; smart ward platform; and
5: visual information terminal.

Specifically, this embodiment uses the medical communication information module 3 as a data acquisition and communication center, integrates and connects informationization systems, such as decentralized and independent mobile nursing of wards, nursing management, ward round, large-screen displaying, call intercom, transfusion monitoring, physical sign data acquisition, staff locating, and accompanying management, to the smart ward platform 4, thereby achieving data fusion and system linkage among multiple systems and solving the problems of independent deployment of multiple systems, complex implementation of wiring, system docking, incapability of sharing data among systems, and the like, which can serve nurses and patients more intelligently and improve the level of informatization. The smart ward platform 4 is connected with a hospital information system, a laboratory information management system, a picture archiving and communication system, and an electronic medical record data interface for data interaction, which can solve the problems of heavy workload and data waste during data connection of multiple systems.

Preferably, the medical information monitoring modules 1 each include a transfusion monitoring unit, an infant anti-theft unit, a physical sign acquisition unit, a staff locating unit, a mobile nursing unit, a nursing management unit, a nursing path unit, a ward round unit, a hospital information unit, a laboratory information management unit, a picture archiving and communication unit, and electronic medical records.

Specifically, the medical related information in this embodiment includes doctor image information, department image information, doctor work situation information, doctor visit information, nurse practitioner image information, nurse practitioner work situation information, and the like.

Preferably, the medical communication information module 3 includes a nurse station, wards, a ward entrance machine, a corridor display screen, a video monitor, and a switch.

Optionally, the patient inquiry information in this embodiment includes patient symptom data and patient feature data.

Furthermore, the patient feature data includes the gender, age, occupation, medical insurance status, annual income, and approved therapy method of a patient.

For example, the patient symptom data can include headache, fever, and edema of lower limbs. The patient feature data can include: gender, for example male; age, for example 35; occupation, for example driver; medical insurance status, for example medical insurance of city a; annual income, for example 50,000-100,000 CNY; and approved therapy method, for example conservative treatment with economical drugs.

Preferably, the visual information terminal 5 includes:
an information extraction unit, configured to perform information screening on the fused information to obtain character picture information in the fused information;
a parsing unit, connected to the information extraction unit and configured to perform three-dimensional parsing on the character picture information to obtain three-dimensional data;
a modeling unit, connected to the parsing unit and configured to build the virtual character model according to the three-dimensional data; and
an information interaction unit, connected to the modeling unit and the information extraction unit and configured to perform human-computer interaction according to the virtual character model and the fused information.

Specifically, the visual information terminal 5 also includes a voice acquisition module, a voice playing module, a memory, a display screen, and an operation inputting apparatus. The voice acquisition module is configured to acquire voice features of various doctors or nurses. After the voice features are acquired, the control unit in the visual information terminal 5 generates corresponding statements according to the voice features and sends the statements to the voice playing module for playing. The memory is configured to store the voice features of the various doctors or nurses.

Preferably, the visual information terminal 5 is any of a mobile terminal, a tablet, or a computer.

As an optional implementation, the visual information terminal 5 in this embodiment can be arranged at an entrance of a department, at an entrance of a hospital, at a bedside, at a nurse station, or in a doctor's office.

The present disclosure has the following beneficial effects.

The present disclosure can perform visual displaying and human-computer interaction according to the obtained information, thereby improving the convenience and efficiency of medical communication.

All the embodiments in the specification are described in a progressive manner. Contents mainly described in each embodiment are different from those described in other embodiments. Same or similar parts of all the embodiments refer to each other.

The principle and implementation modes of the present disclosure are described by applying specific examples herein. The descriptions of the above embodiments are only intended to help to understand the method of the present disclosure and a core idea of the method. In addition, those ordinarily skilled in the art can make changes to the specific implementation modes and the application scope according to the idea of the present disclosure. From the above, the contents of this specification shall not be deemed as limitations to the present disclosure.

What is claimed is:

1. A medical communication system based on an Internet of Things, comprising:
a plurality of medical information monitoring modules, configured to acquire medical relevant information of various departments;
an inquiry information monitoring module, configured to acquire patient inquiry information of various patients making an inquiry;
a medical communication information module, connected to at least one of the plurality of medical information monitoring module and the inquiry information monitoring module, and configured to perform information integration process on the medical relevant information and the patient inquiry information based on the Internet of Things to obtain processed information;
a smart ward platform, connected to the medical communication information module and configured to fuse the processed information to obtain fused information, and perform data interaction with at least one of the plurality of the medical information monitoring modules according to the fused information; and
a visual information terminal, connected to the smart ward platform and configured to extract three-dimensional data in the fused information, and perform character modeling to obtain a virtual character model, and perform human-computer interaction according to the virtual character model and the fused information.

2. The medical communication system according to claim 1, wherein at least one of the plurality of the medical information monitoring modules comprises a transfusion monitoring unit, an infant anti-theft unit, a physical sign acquisition unit, a staff locating unit, a mobile nursing unit, a nursing management unit, a nursing path unit, a ward round unit, a hospital information unit, a laboratory information management unit, a picture archiving and communication unit, and electronic medical records.

3. The medical communication system according to claim 1, wherein the medical communication information module comprises a nurse station, wards, a ward entrance machine, a corridor display screen, a video monitor, and a switch.

4. The medical communication system according to claim 1, wherein the visual information terminal comprises:
an information extraction unit, configured to perform information screening on the fused information to obtain character picture information in the fused information;
a parsing unit, connected to the information extraction unit and configured to perform three-dimensional parsing on the character picture information to obtain three-dimensional data;
a modeling unit, connected to the parsing unit and configured to build the virtual character model according to the three-dimensional data; and
an information interaction unit, connected to the modeling unit and the information extraction unit and configured to perform human-computer interaction according to the virtual character model and the fused information.

5. The medical communication system according to claim 1, wherein the visual information terminal is any of a mobile terminal, a tablet or a computer.
